(19) United States
(12) Patent Application Publication
(10) Pub. No.: US 2013/0084636 A1
(43) Pub. Date: Apr. 4, 2013

(54) SCAFFOLD FOR ARTICULAR CARTILAGE REGENERATION AND METHOD FOR MANUFACTURING SAME

(71) Applicant: Michael Cho, Naperville, IL (US)

(72) Inventor: Michael Cho, Naperville, IL (US)

(73) Assignee: TE BIOS CO., LTD, Chungcheongbuk-do (KR)

(21) Appl. No.: 13/700,972

(22) PCT Filed: Feb. 22, 2011
(86) PCT No.: PCT/KR2011/001133

§ 371 Date: Dec. 5, 2012

(30) Foreign Application Priority Data
Feb. 24, 2010 (KR) 10-2010-0016823

(51) Int. Cl. C12N 5/0775

(52) U.S. Cl. CPC C12N 5/0675; Y10S 977/752; Y10S 977/923; B82Y 5/00
USPC 435/372; 435/366; 977/752; 977/923

(57) ABSTRACT

Disclosed are scaffolds for regeneration of articular cartilage which are applicable to both the superficial zone and the middle zone of articular cartilage, and a method for manufacturing the same. The scaffolds have sufficient mechanical properties to support the implantation and regeneration of chondrocytes, and allow cells to show high cell viability with a high content of sulfated glycosaminoglycans (GAGs). In addition, being applicable to both the superficial zone and the middle zone of articular cartilage, the scaffolds facilitate cell adhesion and provide biomimetic surface environments that are effective for growing and differentiating stem cells. Therefore, the scaffolds are helpful in regenerating damaged articular cartilage, thus finding applications in stem cell therapy for articular cartilage damage and disease. Also, the application of the scaffolds can be extended to prostheses of the ear and the nose in plastic surgery.

### TECHNICAL FIELD

The present invention relates to a scaffold for the regeneration of articular cartilage which is applicable to the middle zone of articular cartilage or both the middle zone and the superficial zone of articular cartilage, and a method for manufacturing the same.

### BACKGROUND ART

Once it is damaged, cartilage, a connective tissue found predominantly in the joints of vertebrates, is hardly apt to regenerate in the body. Persons with damaged articular cartilage can do only limited daily activities because of serious pain they endure. Chronically damaged articular cartilage may be further aggravated and develop into degenerative arthritis, which acts as a serious barrier to physical or vocational activities.

Representative among the therapies for damaged articular cartilage are chondroplasty, osteochondral transplantation, and autologous chondrocyte transplantation.

New tissue engineering-based therapies for damaged articular cartilage have recently gained prominence. Generally, tissue engineering-based therapies employ autologous chondrocytes so as to increase therapeutic effects. After implantation, autologous chondrocytes have relatively high compatibility with normal regions and might be more liable to regenerate free cartilage necessary for joints in practice. However, because chondrocytes are, for the most part, sampled from adults, their growth and proliferation are not very active, which means it takes a significant amount of time to obtain a desired count of chondrocytes ex vivo. Further, mutant phenotypes are sometimes found in ex vivo cultures.

When implanted, mesenchymal progenitors (mesenchymal stem cells (MSCs)), which are undifferentiated cells derived from mesenchymal tissues, such as bone marrow, muscle, skin, etc., are observed to be more apt to proliferate than are differentiated chondrocytes. In fact, unlike tissue structures constructed with differentiated cells, multipotent and non-immunogenic hMSCs exhibit higher cell proliferation and excellent regenerative potential, and therefore, foretell the development of multifunctional tissue scaffolds (such as bone and cartilage tissue), with the reduction or removal of tissue rejection or failure. In addition, hMSCs can be cultured and expanded in vitro and induced by biological or physical stimuli to proliferate and differentiate into tissue-specific cell phenotypes such as chondrogenic cells, osteogenic cells, adipogenic cells, and myogenic cells. Accordingly, hMSCs provide advantages and potential for tissue engineering and the regeneration of articular cartilage.

In tissue engineering for the therapy of articular tissue, scaffolds made of biomaterials occupy an important position. Natural or synthetic biodegradable polymers have been used in tissue engineering based-therapies for articular cartilage. Natural biodegradable polymers available for biomaterials include collagen, alginate, hyaluronic acid, gelatin, chitosan, and fibrin, while synthetic biodegradable polymers may be exemplified by polyglycolic acid (PGA), polylactic acid (PLA), poly(lactic acid-co-glycolic acid) (PLGA), poly-ε-caprolactone (PCL), derivatives thereof, and copolymers thereof. These biomaterials are used to construct various architectures of scaffolds.

Studies have been done on various types of scaffolds, such as hydrogels, nanofibers, beads, spongy materials, etc., for use in therapy for damaged articular cartilage. Hydrogels can facilitate the excretion of the metabolites of the implanted cells and the supply of nutrients and oxygen to the implanted site, and can provide the thickness of a damaged region of articular cartilage. For example, a hydrogel made of collagen type II, a main extracellular matrix component of cartilage, is biocompatible and applicable to articular cartilage. However, collagen-based hydrogels suffer from the drawback of being low in mechanical strength. To overcome this problem, studies have been done in which crosslinkers, such as glutaraldehyde was used to improve the mechanical strength of a hydrogel. The crosslinkers are, however, mostly toxic so it is limited in use.

Since the physical architecture of ECM is in a nano-dimension, biomaterials may be used to fabricate nanofibrous scaffolds, which have high surface-to-volume ratios. Fibers with a nano-dimensional diameter can provide optimal conditions for cell adhesion and growth, and may have influences on cellular activity according to diameter size or fiber direction.

Natural articular cartilage is actually divided into three layers: superficial, middle, and deep zones. These discrete zones are different in organization and function. The superficial zone of natural functioning articular cartilage consists of primarily flattened ellipsoidal-like chondrocytes and a very polarized dense organization of nanoscale collagen type II fibrils. Due to the alignment of chondrocytes and collagen type II fibrils, the thin superficial zone has the greatest tensile strength found in articular cartilage, despite its relatively small thickness (˜200 μm), which is crucial for resisting shear and tensile forces from the articulating surfaces. The middle zone is about 1 mm thick, accounting for 40˜60% of the cartilage thickness, and contains chondrocytes and collagen fibrils, which are non-oriented, unlike the superficial zone.

Previously, scaffolds for the regeneration of articular cartilage have been used in a single zone, particularly the superficial zone, and could not be applied to the middle zone.

Carbon nanotubes are so small in diameter (200˜500 nm) that they can duplicate nanoscale natural ECM well. Their strength is 100-fold greater than that of steel (˜1 TPa), at just ⅙ the weight. In addition, carbon nanotubes are flexible and non-toxic. Also, they are known to be compatible with mammalian cells in natural or synthetic musculoskeletal tissues. Animal tests have shown that carbon nanotubes, although non-biodegradable, do not cause adverse health impacts immediately after injection into the blood stream, but are rapidly removed by the liver or through the renal excretion pathway after circulation. Hence, the recent application of carbon nanotubes to biomaterials for tissue engineering has raised attention. For example, some research reports reveal that the incorporation of carbon nanotubes into tissue engineering biomaterials such as collagen, chitosan, alginate, and hyaluronic acid increases the mechanical properties of the matrix.

Much progress has been made in the research into the fabrication of oriented nanofibers and microfibril scaffolds by electrospinning. Polymeric nanofibers that are provided with orientation by electrospinning can be used to adjust cell direction, allowing tissues to be designed to have optimal functionality. Cells and EMC fibrils in natural tissues are, for the most part, not random, but are well patterned and spatially specific. In addition, a significant improvement in cell adhesion and proliferation is found in oriented nanofibrous scaffolds, compared to randomly oriented nanofibrous scaffolds. Further, fibroblasts cultured on aligned nanofibers are known to secrete a higher level of collagen than are those that are cultured on randomly oriented nanofibers.

Hence, a composite scaffold made of a biomaterial for tissue engineering, such as collagen, and a nanofibrous scaffold can be applied to both superficial and middle zones of articular cartilage if the biomaterial is mechanically strengthened by incorporating carbon nanotubes in it, with the uniform directionality of the nanofibrous scaffold by applying electrospinning. There is, therefore, a pressing need for a scaffold for the regeneration of articular cartilage applicable to both superficial and middle zones of articular cartilage.

### DISCLOSURE

### Technical Problem

The present inventors have studied a scaffold for the regeneration of articular cartilage, applicable to both the superficial zone and the middle zone of articular cartilage, which culminated in finding that when incorporated with multiwalled carbon nanotubes, a 3D collagen type II-based hydrogel can be improved in mechanical properties and can be used as a scaffold for culturing human mesenchymal stem cells, or chondrocytes or osteocytes that are differentiated from human mesenchymal stem cells, with applicability to the middle zone of articular cartilage, that electrospun and biodegradable polymer fibers can be used as an oriented scaffold for culturing human mesenchymal stem cells, or chondrocytes or osteocytes that are differentiated from human mesenchymal stem cells, with applicability to the superficial zone of articular cartilage, and that a composite scaffold prepared by layering the collagen-based hydrogel on the biodegradable polymer fibrous scaffold allows cells to exhibit excellent cell viability, with a high content of sulfated glycosaminoglycans (GAGs).

### Technical Solution

It is an object of the present invention to provide a scaffold for the regeneration of articular cartilage, prepared by seeding human mesenchymal stem cells, or chondrocytes or osteocytes that are differentiated from human mesenchymal stem cells, into multiwalled carbon nanutube-incorporated 3-D collagen type II-based hydrogel, and a method for manufacturing the same.

It is another object of the present invention to provide a composite scaffold for the regeneration of articular cartilage, comprising a scaffold prepared by seeding human mesenchymal stem cells, chondrocytes, or osteocytes that are differentiated from human mesenchymal stem cells into an electrospun and biodegradable polymer, and a collagen gel prepared by seeding human mesenchymal stem cells, or chondrocytes or osteocytes that are differentiated from human mesenchymal stem cells into a multiwalled carbon nanotube-incorporated 3-D collagen type II-based hydrogel, and a method for manufacturing the same.

### DESCRIPTION OF DRAWINGS

FIG. 1 shows scanning electron microscopic (SEM) images of a non-electrospun PCL film (A) and electrospun PCL nanofibers (500 nm in diameter) (B).

FIG. 2 shows confocal microscopic images of the multiwalled carbon nanotube-incorporated 3-D collagen type II-based hydrogel according to the present invention [(A): collagen fibers within collagen hydrogel (blue), (B): multiwalled carbon nanotubes within collagen hydrogel (black)].

FIG. 3 schematically shows the preparation of a composite scaffold comprising (A) an electrospun, biodegradable polymer fibrous scaffold seeded with either human mesenchymal stem cells, or chondrocytes or osteocytes that are differentiated from human mesenchymal stem cells, and (B) a collagen gel composed of a multiwalled carbon nanotube-incorporated 3-D collagen type II-based hydrogel seeded with either human mesenchymal stem cells, or chondrocytes or osteocytes that are differentiated from human mesenchymal stem cells.

FIG. 4 is a graph showing relative physical strengths of a collagen hydrogel, an EDC (1-ethyl-3-(3-dimethylaminopropyl)carbodiimide)-crosslinked collagen hydrogel, and a multiwalled carbon nanotube-incorporated collagen hydrogel, as analyzed by atomic force microscopy (AFM).

FIG. 5 shows the viability and orientation of cells grown on the electrospun PCL fiber scaffold and the non-electrospun PCL film (control scaffold).

FIG. 6 shows the viability and distribution of cells grown on the multiwalled carbon nanotube-incorporated 3D collagen type II-based hydrogel.

FIG. 7 shows contents of sulfated glycosaminoglycans (GAGs) in cells grown on a non-electrospun PCL film (A), an electrospun PCL fibrous scaffold (B), a collagen hydrogel without carbon nanotube (C), and a multiwalled carbon nanotube-incorporated collagen hydrogel (D).

### BEST MODE

In accordance with an aspect thereof, the present invention addresses a scaffold for the regeneration of articular cartilage, comprising a collagen gel composed of a multiwalled carbon nanotube-incorporated 3-D collagen type II-based hydrogel seeded with either human mesenchymal stem cells, or chondrocytes or osteocytes that are differentiated from human mesenchymal stem cells.

In accordance with another aspect thereof, the present invention addresses a method for manufacturing a scaffold for the regeneration of articular cartilage, comprising:

1) preparing a multiwalled carbon nanotube-phosphate buffered saline mixture by primarily ultrasonicating a mixture of multiwall carbon nanotubes, sulfuric acid and nitric acid for 30˜100 min at 30˜70° C., neutralizing the mixture, centrifuging the mixture to collect the multiwalled carbon nanotubes, removing the solvents used, washing the multiwalled carbon nanotubes, secondarily ultrasonicating, recovering the multiwalled carbon nanotubes through centrifugation, and resuspending and dispersing the multiwalled nanotubes in phosphate buffered saline;

2) mixing 70% of collagen type II from articular cartilage, 6.5% of 10× HBSS, 3.5% of 0.4 N NaOH, 1% of 0.4 N acetic acid, and 19% of sterile water to give a collagen hydrogel;

3) combining the multiwalled carbon nanotube-phosphate buffered saline mixture of 1) with the collagen hydrogel of 2), followed by adjusting the combination into a pH of 7˜8 to give a multiwalled carbon nanotube-incorporated 3-D collagen type II-based hydrogel; and

4) seeding and culturing either human mesenchymal stem cells or chondrocytes or osteocytes differentiated from human mesenchymal stem cells in the multiwalled carbon nanotube-incorporated 3-D collagen type II-based hydrogel of 3).

In accordance with a further aspect thereof, the present invention addresses a composite scaffold for the regeneration of articular cartilage, comprising an electrospun and biodegradable polymer fibrous scaffold seeded with either human mesenchymal stem cells, or chondrocytes or osteocytes that are differentiated from human mesenchymal stem cells, and a collagen gel composed of a multiwalled carbon nanotube-incorporated 3-D collagen type II-based hydrogel seeded with either human mesenchymal stem cells, or chondrocytes or osteocytes that are differentiated from human mesenchymal stem cells.

In accordance with a still further aspect thereof, the present invention addresses a method for manufacturing a composite scaffold for the regeneration of articular cartilage, comprising:

1) electrospinning an 8˜15% solution of a biodegradable polymer in an organic solvent at a flow rate of 0.01˜5 mL/h to give an electrospun biodegradable polymer fibrous scaffold; 2) sterilizing the electrospun biodegradable polymer fibrous scaffold by immersing a disc of the electrospun biodegradable polymer fibrous scaffold of 1) in 50˜99% ethanol in a cell culture plate for 30˜100 min, followed by removing the organic solvent in a vacuum chamber for 2˜5 days;

3) immersing the sterilized electrospun biodegradable polymer fibrous scaffold in a complete growth medium supplemented with 15% FBS over the period of 48 hrs, followed by pipetting human mesenchymal stem cells, or chondrocytes or osteocytes that are differentiated from human mesenchymal stem cells onto the electrospun biodegradable polymer fibrous scaffold and by culturing the cells in a complete growth medium over the period of 24 hrs and then in a chondrogenic differentiation medium;

4) preparing a multiwalled carbon nanotube-phosphate buffered saline solution by primarily ultrasonicating a mixture of multiwall carbon nanotubes, sulfuric acid, and nitric acid for 30˜100 min at 30˜70° C., neutralizing the mixture, centrifuging the mixture to collect the multiwalled carbon nanotubes, removing the solvents used, washing the multiwalled carbon nanotubes, secondarily ultrasonicating, recovering the multiwalled carbon nanotubes through centrifugation, and resuspending and distributing the multiwalled nanotubes in phosphate buffered saline;

5) mixing 70% of collagen type II from articular cartilage, 6.5% of 10× HBSS, 3.5% of 0.4 N NaOH, 1% of 0.4 N acetic acid, and 19% of sterile water to give a collagen hydrogel;

6) combining the multiwalled carbon nanotube-phosphate buffered saline solution of 5) with the collagen hydrogel of 5), followed by adjusting the combination to pH of 7˜8 to give a multiwalled carbon nanotube-incorporated 3-D collagen type II-based hydrogel;

7) seeding and culturing either human mesenchymal stem cells or chondrocytes or osteocytes differentiated from human mesenchymal stem cells in the multiwalled carbon nanotube-incorporated 3-D collagen type II-based hydrogel of 6); and

8) pouring the cell-seeded, multiwalled carbon nanotube-incorporated 3-D collagen type II-based hydrogel of 7) onto the cell-seeded electrospun and biodegradable polymer fibrous scaffold of 3) to form a flat layer, followed by allowing the gel to completely set by incubation at 35˜40° C. for 30˜60 min.

Below, a detailed description will be given of the present invention.

The present invention pertains to a scaffold for the regeneration of articular cartilage, comprising a collagen gel prepared by seeding either human mesenchymal stem cells, or chondrocytes or osteocytes that are differentiated from human mesenchymal stem cells, into a multiwalled carbon nanotube-incorporated 3-D collagen type II-based hydrogel, which is applicable to the middle zone of articular cartilage.

Also, the present invention pertains to a composite scaffold for the regeneration of articular cartilage, comprising a scaffold prepared by seeding human mesenchymal stem cells into an electrospun and biodegradable polymer fibrous scaffold, and a collagen gel prepared by seeding human mesenchymal stem cells into a multiwalled carbon nanotube-incorporated 3-D collagen type II-based hydrogel, which is applicable to both the superficial zone and the middle zone of articular cartilage.

Examples of the biodegradable polymer include polyglycolic acid (PGA), polylactic acid (PLA), poly(lactic-co-glycolic acid) (PLGA), poly-ε-caprolactone(PCL), polyanhydride, polyorthoesters, polyvinylalcohol, polyethylene glycol, polyurethane, polyacrylic acid, poly-N-isopropyl acrylamide, poly(ethylene oxide)-poly(propylene oxide)-poly(ethylene oxide)copolymers, derivatives thereof, and copolymers thereof, but are not limited thereto.

The human mesenchymal stem cells usable in the present invention are preferably bone marrow-derived human mesenchymal stem cells, but are not limited thereto.

The collagen gel used as a scaffold for the regeneration of articular cartilage in accordance with the present invention may be manufactured as follows. First, multiwalled carbon nanotubes are mixed in sulfuric acid and nitric acid, ultrasonicated for 30˜100 min at 30˜70° C. in an ultrasonic bath, neutralized, and centrifuged to collect the multiwalled carbon nanotubes. After removal of the solvents, the multiwalled carbon nanotubes are washed with sterile water, and ultrasonicated, followed by centrifugation. The multiwalled carbon nanotube pellets are resuspended and dispersed in phosphate buffered saline to give a multiwalled carbon nanotubes-phosphate buffered saline solution.

Separately, a collagen hydrogel is prepared by mixing 70% of collagen type II (10 mg/mL in 0.02 N acetic acid), 6.5% of 10× HBSS, 3.5% of 0.4 N NaOH, 1% of 0.4 N acetic acid, and 19% of sterile water. Then, the multiwalled carbon nanotubes-phosphate buffered saline mixture is combined into so as to improve the mechanical properties of the collagen hydrogen. Subsequently, either human mesenchymal stem cells, or chondrocytes or osteocytes that are differentiated from human mesenchymal stem cells, are seeded into and cultured in the multiwalled carbon nanotube-incorporated 3-D collagen type II-based hydrogel.

In the multiwalled carbon nanotube-incorporated 3-D collagen type II-based hydrogel, the multiwalled carbon nanotubes help collagen fibers form uniformly over the scaffold. In addition, cell viability was measured to be excellent in both 3-D collagen type II-based hydrogels incorporated with and without multiwalled carbon nanotubes, indicating that the presence of multiwalled carbon nanotubes in 3-D collagen type II-based hydrogel has negative influences on neither the cell viability nor the distribution of the cells cultured therein. Moreover, a higher content of sulfated glycosaminoglycans (GAGs) in cells was measured in the incorporated collagen hydrogel than in a multiwalled carbon nanotube-free collagen hydrogel.

The composite scaffold for the regeneration of articular cartilage in accordance with the present invention is manufactured as follows.

Steps 1) to 3) are adapted to provide an electrospun and biodegradable polymer fibrous scaffold into which human mesenchymal stem cells are seeded.

First, a biodegradable polymer is dissolved in an organic solvent to give an 8˜15%, preferably 10% polymer solution. Then, the polymer solution is electrospun at a flow rate of 0.01˜5 mL/h, and preferably at a flow rate of 1 mL/h, to a rotary aluminum disk collector located 120 mm away from the spinneret to give a biodegradable polymer fiber as a scaffold. For the electrospinning, an electric field is preferably set at 0.1˜10 kV/cm. The resulting biodegradable polymer-electrospun scaffold disk is placed on a cell culture plate, immersed in 50˜99% ethanol for 30˜100 min, and dried in a vacuum chamber to remove any organic solvent which might remain, followed by UV sterilization. The sterilized electrospun biodegradable polymer fibrous scaffold is placed in a complete growth medium (supplemented with 15% FBS) over the period of 48 hrs before cell seeding. Subsequently, human mesenchymal stem cells, or chondrocytes or osteocytes that are differentiated from human mesenchymal stem cells, were pipetted onto the scaffold, and cultured over the period of 24 hrs in the complete growth medium, and then in a chondrogenic differentiation medium.

The organic solvent may include at least one selected from the group consisting of methylene chloride, dimethyl formamide, hexane, chloroform, acetone, dioxane, tetrahydrofuran, and hexafluoroisopropane, but is not limited thereto.

A unidirectional orientation is found in the electrospun biodegradable polymer fibrous scaffold, but not in the non-electrospun biodegradable polymer films which are randomly oriented. In addition, cells show higher viability with a higher content of sulfated glycosaminoglycans (GAGs) when cultured on the electrospun biodegradable polymer fibrous scaffold than on the non-electrospun biodegradable polymer film.

In steps 4) to 7), either human mesenchymal stem cells, or chondrocytes or osteocytes that are differentiated from human mesenchymal stem cells, are seeded into a multiwalled carbon nanotube-incorporated 3-D collagen type II-based hydrogel to prepare a collagen gel. Steps 4)˜7) comprise the same procedure that is described for the method for manufacturing a scaffold for the regeneration of articular cartilage.

Step 8) is adapted to prepare a bilayer composite scaffold. For this, the multiwalled carbon nanotube-incorporated 3-D collagen type II-based hydrogel in which cells have been cultured is poured onto the electrospun and biodegradable polymer fibrous scaffold in which cells have been cultured, followed by incubation at 35˜40° C. for 30˜60 min to completely set the gel to afford a bilayer composite scaffold.

The composite scaffold composed of an electrospun biodegradable polymer fibrous scaffold/a multiwalled carbon nanotube-incorporated 3-D collagen type II-based hydrogel is observed to have excellent physical properties in terms of complex viscosity, storage modulus, loss modulus, and loss factor, and to guarantee high cell viability and a high total count of stem cells per area.

As described above, the scaffold for the regeneration of articular cartilage in accordance with the present invention has sufficient mechanical properties to implant and regenerate cartilage, and allows cells to be highly viable with a high content of sulfated glycosaminoglycans (GAGs). In addition, the scaffold of the present invention is specifically applicable to the superficial zone and the middle zone of articular cartilage, providing a biomimetic surface environment that is effective for growing and differentiating stem cells. The scaffold of the present invention is effective in the regeneration of damaged articular cartilage and is thus also effective in stem cell therapy for articular cartilage damage and diseases. Also, it finds applications in prostheses of the ear and nose in plastic surgery.

The articular cartilage diseases to which the scaffold of the present invention can be therapeutically applicable include degenerative arthritis, rheumatoid arthritis, bone fracture, muscular tissue injury, plantar fasciitis , lateral epicondylitis, calcific tendinitis, nonunion of fracture, and traumatic joint injury, but is not limited thereto.

### Mode for Invention

A better understanding of the present invention may be obtained through the following examples which are set forth to illustrate, but are not to be construed as limiting, the present invention.

### EXAMPLE 1

### Preparation of Scaffold Composed of Electrospun Poly-ε-Caprolactone (PCL) Fiber Scaffold Seeded with Human Mesenchymal Stem Cells

1. Electrospinning of Poly-ε-Caprolactone (PCL) into Fiber Scaffold

Oriented PCL fiber scaffolds were prepared using an electrospinning method as reported previously [Reneker, D. H., Yarin, A. L., Fong, H., Koombhongse, S.: Bending instability of electrically charged liquid jets of polymer solutions in electrospinning. J. App. Phys., 87: 4531, 2000.; Theron, A., Zussman, E., Yarin, A. L.: Electrostatic field-assisted alignment of electrospun nanofibres. Nanotechnology, 12: 384, 2001; Zussman et al., 2003]. In detail, PCL with a molecular weight of 80 kDa (Sigma-Aldrich, St. Louis, Mo.) was dissolved in a mixed solvent of methylene chloride/dimethyl formamide (75/25(vol.)) to give a 10% PCL solution. The 10% PCL solution was electrospun from a 5 mL hypodermic syringe needle (0.1 mm in inner diameter) at a flow rate of 1 mL/h to a rotating aluminum disk collector located 120 mm away from the spinneret. For this, an electric field was set at 1.1 kV/cm, with the linear velocity of the rotating disc collector at the edge given 10 m/s. During electrospinning, fibers were formed on the table (5×4 mm) placed on the keen edge of the rotating aluminum disc collector, so that they were definitely oriented in the rotational direction of the disc.

A non-electrospun, porous PCL film was prepared and used as a control. In this regard, the 10% PCL solution was poured into a 1 mm-thick flat-bottom mold and the solvent was evaporated to form a non-electrospun, porous PCL film which was withdrawn from the mold. All experiments were carried out at room temperature (about 25° C.) under an air circulation condition with a relative humidity of 40%.

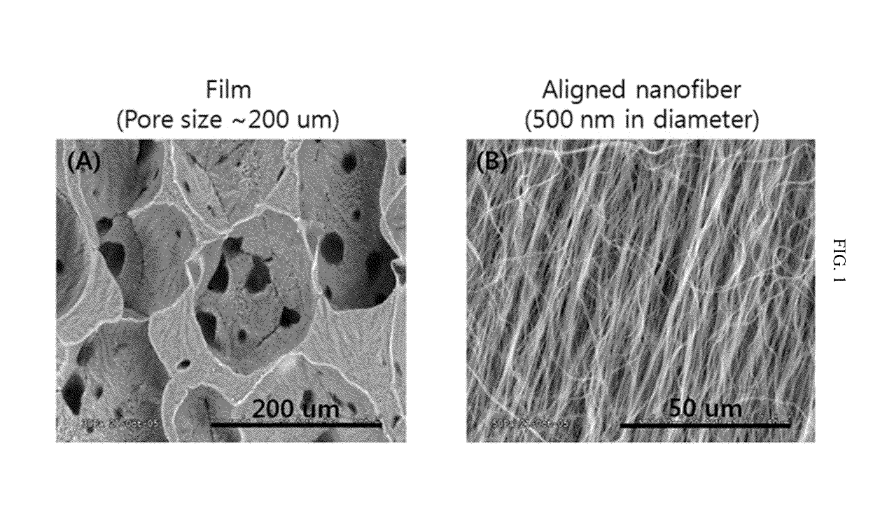

FIG. 1 shows scanning electron microscopic (SEM) images of the non-electrospun PCL film (A) and the electrospun PCL nanofibers (500 nm in diameter) (B).

As can be seen in FIG. 1, the electrospun PCL nanofibers (500 nm in diameter) had constant orientation, whereas the non-electrospun PCL film was non-oriented.

2. Seeding of Human Mesenchymal Stem Cells into the Electrospun Poly-ε-Caprolactone (PCL) Fiber Scaffold

The electrospun PCL fiber scaffold prepared in 1 was cut into a disc (about 2 cm²) which was then placed on a 24-well plate for cell culture. The scaffold was immersed for 1 hr in 70% ethanol and placed for 3 days in a vacuum chamber to remove residual organic solvents, followed by UV sterilization for 6 hrs. To promote protein adsorption and cell adhesion, the PCL fiber scaffold was immersed in a complete growth medium (supplemented with 15% FBS) for 48 hrs before cell seeding. Human mesenchymal stem cells (hMSCs) were directly pipetted at a density of 6×10⁴ cells/cm² onto the electrospun PCL fiber scaffold or the non-electrospun PCL film (control scaffold) and cultured in the complete growth medium. For chondrogenesis, the culture medium was replaced by a chondrogenic differentiation medium [4,500 mg/L D-glucose, L-glutamine, and 110 mg/L sodium pyruvate, Invitrogen] containing 10 ng/mL TGF-β1 (Research Diagnostics, Inc.), 100 nM dexamethasone (Sigma), 50 μg/mL ascorbate 2-phosphate (Sigma), 40 pg/mL proline (Sigma), 1% broth supplement (ITS+1, Sigma, containing 5 μg/mL insulin, 5 μg/mL transferrin, and 5 ng/mL selenious acid), abd 1% antibiotics, and antifungal agents (final concentrations: penicillin 100 units/mL, streptomycin 100 mg/mL, and amphotericin B 0.25 mg/mL). The cells were cultured on the electrospun PCL fiber scaffold or the non-electrospun PCL film (control) for 35 days, with the complete growth medium or the chondrogenic differentiation medium replaced by a fresh one every two or three days.

### EXAMPLE 2

### Preparation of Collagen Gel Composed of Multiwalled Carbon Nanotube-Incorporated 3-D Collagen Type II-Based Hydrogel Seeded with Human Mesenchymal Stem Cells

1. Multiwalled Carbon Nanotubes (MWCNT)-PBS Solution

To a mixture of 15 mL of sulfuric acid and 5 mL of nitric acid, 50 mg of MWCNT [240˜500 nm in outer diameter, 5˜40 ∥m in length, 95+% in purity, manufactured by catalytic chemical vapor deposition (CVD), Nanostructured and Amorphous Materials Inc.] was added, and the solution was ultrasonicated for 1 hr at 50° C. in an ultrasonication water bath, and neutralized with ammonium hydroxide. The MWCNT was collected as pellets by centrifugation for 10 min at 5,000 rpm, and the supernatant was removed. The pellets were washed four times with sterile water, ultrasonicated for 15 min, and centrifuged. After removal of the supernatant containing residual solvents, and undesired amorphous carbon, the MWCNT was resuspended and dispersed in 4 mL of phosphate buffered saline (PBS) to give a 6 mg/mL MWCNT-PBS suspension.

2. Preparation of 3 D Collagen Type II-Based Hydrogel

3-D collagen type II-based hydrogel was synthesized using a modified version of the method disclosed in the following literature [Sun, S., Wise, J., Cho, M.: Human fibroblast migration in three-dimensional collagen gel in response to noninvasive electrical stimulus: characterization of induced three-dimensional cell movement. Tissue Eng., 10: 1548, 2004]. In detail, a 1 mL collagen hydrogel was prepared by mixing 700 μL of bovine collagen type II (10 mg/mL in 0.02 N acetic acid) (Elastin Products, Inc) (70%), 66.5 μL of 10× HBSS (Hanks balanced salt solution, Sigma) (6.5%), 33.5 μL of 0.4 N NaOH (Sigma) (3.5%), 10 μL of 0.4 N acetic acid (Sigma) (1%), and 190 μL of sterile water (Sigma) (19%),. The acidity of the collagen hydrogel was adjusted to pH of about 7.5 by dropwise adding of 3 μL of 1 N NaOH. The 3-D collagen type II-based hydrogel had a final concentration of 7 mg/mL. For experiments with cells, 3-D collagen type II-based hydrogel discs (1 cm² in surface area, 2 mm in thickness) were created by pipetting the 3-D collagen type II-based hydrogel into sterilized well-plates, and incubated at 37° C. for 30 min before application to a growth medium.

3. Incorporation of Multiwalled Carbon Nanotubes into 3-D Collagen Type II-Based Hydrogel

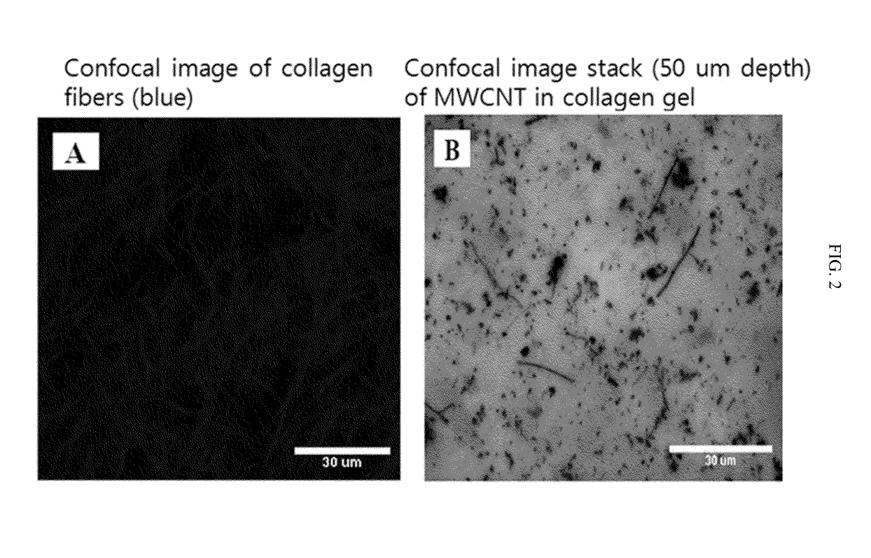

To 1 mL of the hydrogel prepared in 2, 90 μL of the ultrasonicated 6 mg/mL MWCNT-PBS suspension prepared in 1 was added (to form a final concentration of 0.5 mg/mL of MWCNT in the hydrogel), and the pH of the resulting solution was adjusted to 7.5. The multiwalled carbon nanotube-incorporated 3 D collagen type II-based hydrogel thus obtained was observed under a confocal microscope, and the images are given in FIG. 2.

As can be seen in FIG. 2, the multiwalled carbon nanotubes (black) were formed evenly over the 3-D collagen type II-based hydrogel, without interfering with the formation of collagen fibers (blue).

4. Seeding of Human Mesenchymal Stem Cells into Multiwalled Carbon Nanotube-Incorporated 3-D Collagen Type II-Based Hdrogel

Human mesenchymal stem cells (hMSCs) were seeded at a density of 8×10⁴ cells/mL into 1 mL of the multiwalled carbon nanotube-incorporated 3-D collagen type II-based hydrogel prepared in 3 and cultured, followed by pH adjustment to 7.5.

### EXAMPLE 3

### Preparation of Composite Scaffold Composed of Electrospun PCL Fiber Scaffold/Multiwalled Carbon Nanotube-Incorporated 3-D Collagen Type II-Based Hydrogel

The multiwalled carbon nanotube-incorporated 3-D collagen type II-based hydrogel prepared in Example 2 was pipetted in an amount of 400 μL onto the electrospun PCL fiber scaffold prepared in Example 1 and applied to the flat bottom of each well of 24-well plates to form 2 mm-thick constructs. Subsequently, they were completely set by incubation at 37° C. for 45 min to afford a bilayer composite scaffold of 2 mm in thickness in which the thin PLC fiber scaffold was firmly incorporated onto one side of the completely solidified 3-D collagen type II-based hydrogel. This composite scaffold was withdrawn from the 24-well plate, transferred into a petri dish filled with sterile water, prior to maintaining hydration with sterile water and AFM analysis. It was incubated at 37° C. for 30 min before AFM (atomic force microscope) analysis.

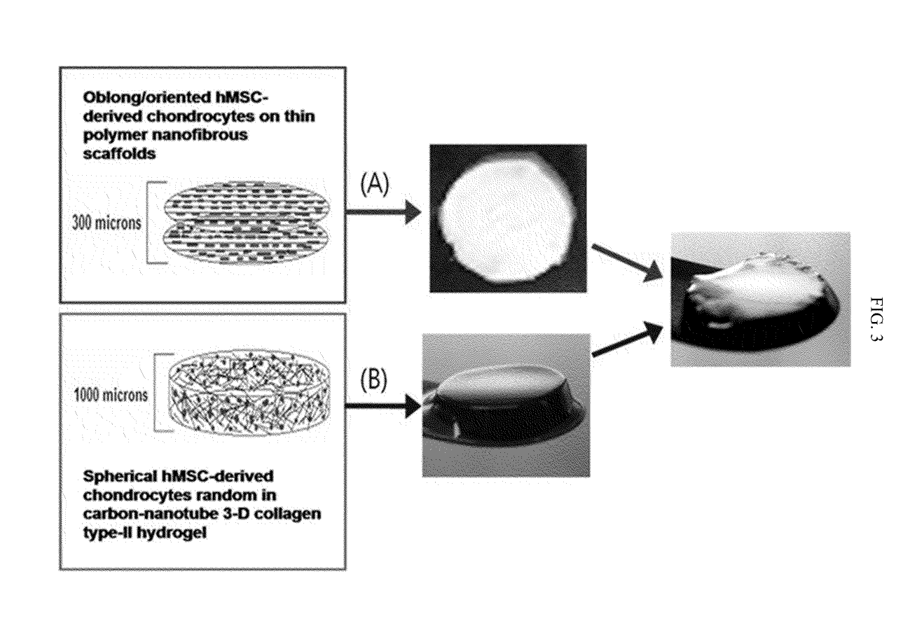

FIG. 3 schematically shows the preparation of a composite scaffold comprising (A) an electrospun, biodegradable polymer fibrous scaffold seeded with either human mesenchymal stem cells, or chondrocytes or osteocytes that are differentiated from human mesenchymal stem cells, and (B) a collagen gel composed of a multiwalled carbon nanotube-incorporated 3-D collagen type II-based hydrogel seeded with either human mesenchymal stem cells, or chondrocytes or osteocytes that are differentiated from human mesenchymal stem cells.

### EXPERIMENTAL EXAMPLE 1

### Physical Strength of 3-D Collagen Type II-Based Hydrogel

The physical strength of the 3-D collagen type II-based hydrogel was measured by AFM (atomic force microscopy). None of the hydrogel samples analyzed by AFM contained cells.

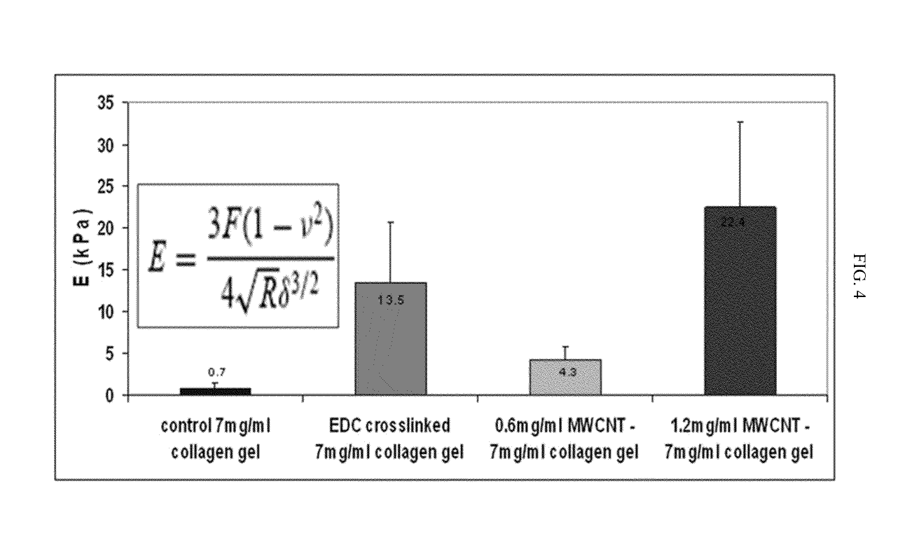

The ultrasonicated and multiwalled carbon nanotubes were added directly within 1 mL of the collagen hydrogel with pH of 7.5. For comparison, the control collagen hydrogel, EDC (1-ethyl-3-(3-dimethylaminopropyl)carbodiimide)-crosslinked collagen hydrogel, and multiwalled carbon nanotube-incorporated collagen hydrogel were employed. All samples were adjusted to have a final collagen concentration of 7 mg/mL. Samples were created by pipetting 50 μL of the multiwalled carbon nanotube-incorporated collagen hydrogel preparation onto a glass cover slip, and allowing the gel to set completely by incubating at 37° C. for 30 min. The collagen hydrogel samples were kept hydrated in sterile water prior to AFM analysis. AFM analysis was performed with an Atomic Force Microscope (Novascan Technologies, Ames, Iowa) mounted on an inverted Nikon microscope. Silicon nitride (Si₃N₄) cantilevers, each 100 μm long, were employed. For control collagen gel samples, a 0.12 N/m (Modulus of Elasticity, k) silicon nitride cantilever was used, and for the EDC-crosslinked collagen gel sample and the multiwalled carbon nanotubes-incorporated collagen gel sample, a 0.32 N/m (Modulus of Elasticity, k) silicon nitride cantilever was used. Borosilicate glass beads with a diameter of 10 μm glued onto the cantilever served as collagen-based gel indenters. The force curve was obtained by measuring the cantilever deflection at every vertical z-position of the cantilever. The force distance curves were collected and analyzed according to the Hertz model. For each sample, the average Young's modulus was calculated from the force-indentation data using the Hertz model for spherical probe according to the following equation. The results are depicted in FIG. 4.

$$E = \frac{3 F ( 1 - {v}^{2} )}{4 \sqrt{R} {δ}^{\frac{3}{2}}}$$

wherein,

F: applied nanomechanical load,

v: estimated Poisson's ratio for a given region,

R: radius of curvature of the spherical indenter,

δ: amount of indentation to the sample.

As can be seen in FIG. 4, the strength of the hydrogel was about 22-fold increased when it was incorporated with 1.2 mg/mL multiwalled carbon nanotubes, compared to that of the control hydrogel. Particularly, the strength of the multiwalled carbon nanotube-incorporated collagen hydrogel was twice as high as that of the EDC-crosslinked collagen hydrogen.

### EXPERIMENTAL EXAMPLE 2

### Physical Properties of the Composite Scaffold

The composite scaffold of the present invention was examined for physical properties using a HAAKE RheoStress 1 Rotational Rheometer (Thermo Scientific) equipped with two parallel plates of 2 cm diameter.

In detail, a scaffold for the regeneration of articular cartilage comprising the multiwalled carbon nanotube-incorporated 3-D collagen type II-based hydrogel prepared in Example 2, and a composite scaffold for the regeneration of articular cartilage comprising the electrospun PCL fiber scaffold/multiwalled carbon nanotube-incorporated 3-D collagen type II-based hydrogel prepared in Example 3 were used as samples. Discs of these samples were placed between the two parallel plates of 2 cm diameter, and tested using the oscillation mode at frequencies of either 0.6 Hz or 2 Hz to obtain data for complex viscosity, storage modulus, loss modulus, and loss factor. In the oscillation mode, the linear viscosity-elasticity range of recommended frequencies is from approximately 0.01 to 10 Hz. The complex viscosity is the ratio of the complex shear modulus to the oscillation frequency in rad/sec. The storage modulus (G′) reflects the elastic property of the material, and more specifically it is the ratio of elastic peak amplitude shear stress to peak amplitude shear strain for the torque component in phase with a sinusoidally applied strain. The loss modulus (G″) reflects the viscous property of the material, and more specifically it is the ratio of viscous peak amplitude shear stress to peak amplitude shear strain for the torque component at 90° out of phase with a sinusoidally applied strain. The loss factor, which can also be referred to as a damping factor, is the ratio of loss modulus to storage modulus, or the ratio of viscous torque to elastic torque.

The results are summarized in Table 1, below.

TABLE 1

| | | Com- | | | |
|---|---|---|---|---|---|
| | | plex | | | |
| | | Visc. | Storage | Loss | Loss |
| | Freq. | (η* | Modulus | Modulus | Factor |
| Sample | [Hz]) | [cP]) | (G′ [Pa]) | (G″ [Pa]) | (tan [δ]) |
| Scaffold for regenerating | 0.6 | 16876 | 68 | 14 | 0.20 |
| articular cartilage, | 2 | 6122 | 78 | 3 | 0.04 |
| comprising a multiwalled | | | | | |
| carbon nanotube-incor- | | | | | |
| porated | | | | | |
| 3-D collagen type II-based | | | | | |
| hydrogel | | | | | |
| Composite scaffold, | 0.6 | 53530 | 211 | 43 | 0.20 |
| comprising electro- | | | | | |
| spun PCL | | | | | |
| fiber scaffold/multiwalled | 2 | 15714 | 210 | 52 | 0.25 |
| carbon nanotube-incor- | | | | | |
| porated | | | | | |
| 3-D collagen type II-based | | | | | |
| hydrogel | | | | | |

As is understood from the data of Table 1, the composite scaffold for the regeneration of articular cartilage comprising the electrospun PCL fiber scaffold/multiwalled carbon nanotube-incorporated 3-D collagen type II-based hydrogel is superior in terms of complex viscosity, storage modulus, loss modulus, and loss factor to the scaffold for the regeneration of articular cartilage comprising the multiwalled carbon nanotube-incorporated 3-D collagen type II-based hydrogel. Accordingly, biomimetic scaffolds for articular cartilage have higher physical properties when fabricated into composites than single layer structures.

### EXPERIMENTAL EXAMPLE 3

### Cell Viability and Cell Orientation on Electrospun PCL Fiber Scaffold

The following experiments were carried out to examine cell viability and cell orientation on the electrospun PCL fiber scaffold.

1. Cell Viability on Electrospun PCL Fiber Scaffold

To examine cell viability on the electrospun PCL fiber scaffold prepared in Example 1, cells were stained (Molecular Probes, Carlsbad, Calif.). For this, live cells were fluorescently stained with calcein AM (calcein acetomethylester) while dead cells with damaged membranes were stained with 4 mM ethidium homodimer-1 before microscopic observation. Calcein AM diffuses across the cell membrane of live cells and reacts with intracellular esterase to produce green fluorescence. On the other hand, ethidium homodimer-1 enters cells through damaged cell membranes and is bound to nucleic acids to produce red fluorescence.

2. Cell Orientation on Electrospun PCL Fiber Scaffold

Cells grown on the electrospun PCL fiber scaffold or the non-electrospun PCL film (control scaffold) in a complete growth medium or a chondrogenic differentiation medium were examined for orientation.

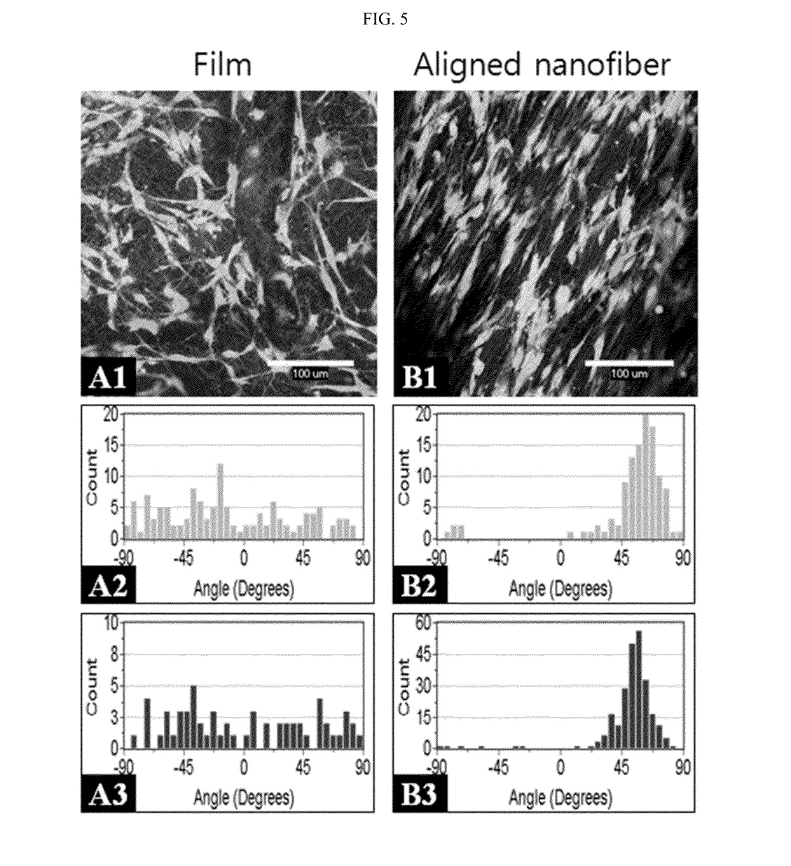

After culturing for 4 and 18 days on the electrospun PCL fiber scaffold and the non-electrospun PCL film (control scaffold), cell viability and total stem cell counts were measured and are summarized in Table 2 below. Observations of the viability and orientation of cells grown on the electrospun PCL fiber scaffold and the non-electrospun PCL film (control scaffold) are given in FIG. 5.

TABLE 2

| | | Cell | Total Count of |
|---|---|---|---|
| Time Period | PCL Fiber | Viability | Stem Cells |
| of Culture | Scaffold | (%) | (cells/cm²) |
| 4 Days | Non-Electrospun | 76 | 7 × 10⁴ |
| | Electrospun | 80 | 11.6 × 10⁴ |
| 18 Days | Non-Electrospun | 73 | 32.9 × 10⁴ |
| | Electrospun | 76 | 20.2 × 10⁴ |

As is understood from the data of Table 2, the electrospun PCL fiber scaffold guaranteed higher cell viability and total stem cell counts per unit area than did the non-electrospun PCL film (control scaffold).

In addition, microscopic images of FIG. 5 demonstrate that the electrospun PCL fiber scaffold is superior in cell viability to the non-electrospun PCL film (control scaffold). Cell orientation was in a constant direction on the electrospun PCL fiber scaffold while being random on the non-electrospun PCL film (control scaffold). Therefore, the electrospun PCL fiber scaffold is deemed well applicable to the superficial zone of articular cartilage.

### EXPERIMENTAL EXAMPLE 4

### Cell Viability and Distribution on Multiwalled Carbon Nanotube-Incorporated 3-D Collagen Type II-Based Hydrogel

Cell viability and distribution on the multiwalled carbon nanotube-incorporated 3-D collagen type II-based hydrogel of the present invention were examined as follows.

Cells on the multiwalled carbon nanotube-incorporated 3-D collagen type II-based hydrogel prepared in Example 2 were cultured in a complete growth medium or a chondrogenic differentiation medium for 21 days, and then to day 35 with the complete growth medium or chondrogenic differentiation medium replaced by a fresh one every two or three days. Cell viability on the multiwalled carbon nanotube-incorporated 3-D collagen type II-based hydrogel was evaluated with a staining assay. For comparison, a 3-D collagen type II-based hydrogel free of multiwalled carbon nanotubes was used.

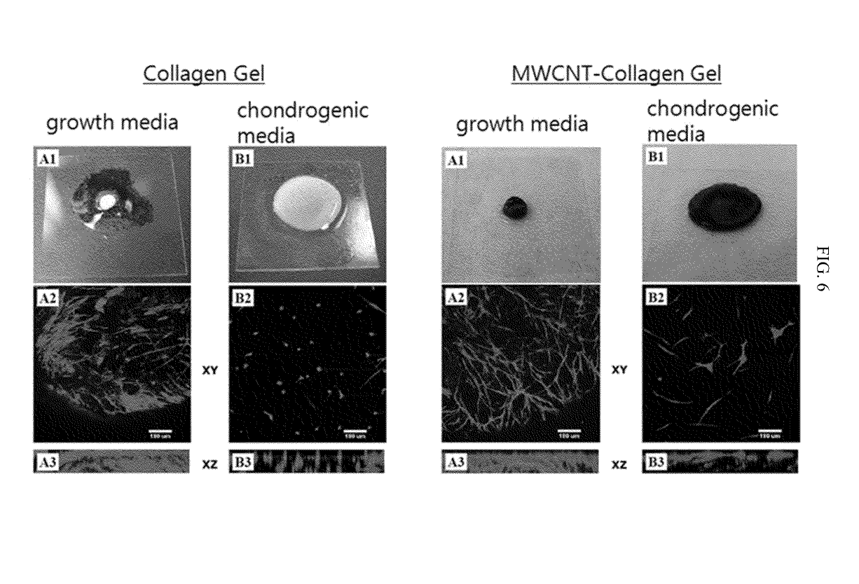

The results are shown in FIG. 6.

Both the multiwalled carbon nanotube-incorporated 3-D collagen type II-based hydrogel and the multiwalled carbon nanotube-free 3-D collagen type II-based hydrogel, as shown in FIG. 6, allowed excellent cell viability (green fluorescence), indicating that the multiwalled carbon nanotubes, when combined with a 3-D collagen type II-based hydrogel, has no negative influence on cell viability and distribution.

### EXPERIMENTAL EXAMPLE 5

### Content of Sulfated Glycosaminoglycans (GAGs) in Cells Grown on Multiwalled Carbon Nanotube-Incorporated 3-D Collagen Type II-Based Hydrogel

Cells grown the multiwalled carbon nanotube-incorporated 3-D collagen type II-based hydrogel were examined for content of sulfated glycosaminoglycans (GAGs) as follows.

GAG production is the marker of chondrogenesis, and the content of GAGs may serve as a reference for evaluating cartilage regeneration. Cells were seeded at a density of 6×10⁴ cells/cm² onto the non-electrospun PCL film or the electrospun PCL fiber scaffold, and at a density of 8×10⁴ cells/mL into the multiwalled carbon nanotube-void collagen hydrogel or the multiwalled carbon nanotube-incorporated collagen hydrogel, and cultured in a typical growth medium or a chondrogenic differentiation medium. Sulfated GAGs and DNA quantitation was performed at time points of day 1 and day 34 for the non-electrospun PCL film and the electrospun PCL fiber scaffold and at time points of day 1 and day 24 for the multiwalled carbon nanotube-void collagen hydrogel and the multiwalled carbon nanotube-incorporated collagen hydrogel. DNA and GAGs were extracted from all samples and quantitatively analyzed. In this regard, a solution of papain, EDTA, PBS, and DTT was used to extract GAGs and DNA. Specifically, each cell-seeded sample was digested in 100 μL of a solution of 300 μg/mL papain in 20 mM PBS, 5 mM EDTA, and 2 mM DTT at 60° C. for 18 hrs.

For total GAGs quantitation, Blyscan™ Sulfated Glycosaminoglycan Assay Kit (Biocolor, N. Ireland) was employed. Briefly, 1 mL of DMB (1,9-dimethylmethylene blue) dye reagent was added to 50 μL of the extract for each sample and allowed to react for 30 min. The blue dye binds to GAGs and forms a purple dye-GAGs precipitate, which was separated from the unbound dye solution by centrifugation at 10,000 g. To recover the GAGs-bound dye from the resulting pellet, 200 μL of dissociation reagent was added. Absorbance of dye from GAGs samples was quantified spectrophotometrically with a 655 nm filter on a Model 680 Microplate Reader (Bio-Rad Laboratories, Hercules, Calif.).

For total DNA analysis, a fluorescent DNA Quantitation Kit (Bio-Rad Laboratories, Hercules, Calif., Catalog #170-2480) was employed. Briefly, 20 μL from the remaining 50 μL of the DNA/GAGs extract was added to 80 μL of 1 μg/mL Hoechst 33258 dye. The fluorescence of the Hoechst 33258-DNA complex was detected at an excitation/emission wavelength of 360 nm/460 nm using a SpectraMax Gemini Microplate Spectrofluorometer (Molecular Devices, Sunnyvale, Calif.). Ratios of GAGs to total DNA for each cell-seeded sample were determined.

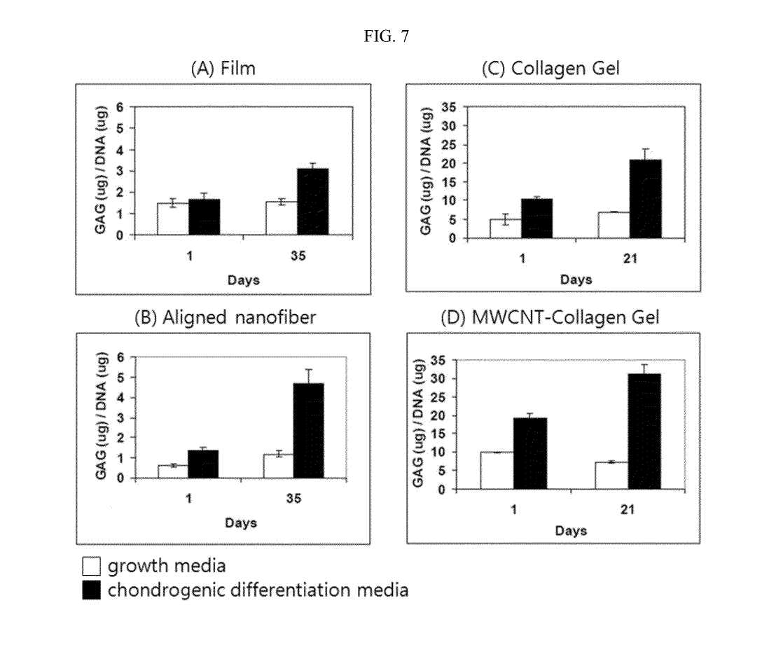

The results are shown in FIG. 7.

As is apparent from FIG. 7, higher contents of sulfated GAGs were detected in the cells grown on the electrospun PCL fiber scaffold than on the non-electrospun PCL film, and in the cells grown on the multiwalled carbon nanotube-incorporated collagen hydrogel than on the multiwalled carbon nanotube-void collagen hydrogel. Individual single layers made of the electrospun PCL fiber scaffold or the multiwalled carbon nanotube-incorporated collagen hydrogel, which are respectively applicable to the superficial zone and the middle zone of articular cartilage, exerted higher effects on chondrogenesis than did the corresponding controls. Accordingly, these single layers may exhibit a synergistic effect when they are combined into a composite scaffold.

### INDUSTRIAL APPLICABILITY

As delineated hitherto, the scaffolds for the regeneration of articular cartilage in accordance with the present invention have mechanical properties that are sufficient to support the implantation and regeneration of chondrocytes, and allow cells to show high cell viability with a high content of sulfated glycosaminoglycans (GAGs). In addition, being applicable to both the superficial zone and the middle zone of articular cartilage, the scaffolds facilitate cell adhesion and provide biomimetic surface environments that are effective for growing and differentiating stem cells. Therefore, the scaffolds for the regeneration of articular cartilage in accordance with the present invention are helpful in regenerating damaged articular cartilage, thus finding applications in stem cell therapy for articular cartilage damage and disease. Also, the application of the scaffolds can be extended to the prosthesis of the ear and the nose in plastic surgery.

We claim,:

1. A scaffold for regeneration of articular cartilage, comprising collagen gel consisting of a multiwalled carbon nanotube-incorporated 3-D collagen type II-based hydrogel seeded with either human mesenchymal stem cells, or chondrocytes or osteocytes that are differentiated from human mesenchymal stem cells.

2. The scaffold of claim 1, wherein human mesenchymal stem cells are derived from bone marrow.

3. A method for manufacturing the scaffold of claim 1, comprising:
1) preparing a multiwalled carbon nanotube-phosphate buffered saline mixture by primarily ultrasonicating a mixture of multiwall carbon nanotubes, sulfuric acid, and nitric acid for 30˜100 min at 30˜70° C., neutralizing the mixture, centrifuging the mixture to collect the multiwalled carbon nanotubes, removing the solvents used, washing the multiwalled carbon nanotubes, secondarily ultrasonicating, recovering the multiwalled carbon nanotubes through centrifugation, and resuspending and dispersing the multiwalled nanotubes in phosphate buffered saline;
2) mixing 70% of collagen type II from articular cartilage, 6.5% of 10× HBSS, 3.5% of 0.4 N NaOH, 1% of 0.4 N acetic acid, and 19% of sterile water to give a collagen hydrogel;
3) combining the multiwalled carbon nanotube-phosphate buffered saline mixture of 1) with the collagen hydrogel of 2), followed by adjusting the mixture to pH of 7˜8 to give a multiwalled carbon nanotube-incorporated 3-D collagen type II-based hydrogel; and
4) seeding and culturing either human mesenchymal stem cells or chondrocytes or osteocytes differentiated from human mesenchymal stem cells in the multiwalled carbon nanotube-incorporated 3-D collagen type II-based hydrogel of 3).

4. The method of claim 3, wherein the human mesenchymal stem cells are derived from bone marrow.

5. A composite scaffold for regeneration of articular cartilage, comprising an electrospun and biodegradable polymer fibrous scaffold seeded with either human mesenchymal stem cells, or chondrocytes or osteocytes that are differentiated from human mesenchymal stem cells, and a collagen gel composed of a multiwalled carbon nanotube-incorporated 3-D collagen type II-based hydrogel seeded with either human mesenchymal stem cells, or chondrocytes or osteocytes that are differentiated from human mesenchymal stem cells.

6. The composite scaffold of claim 5, wherein the biodegradable polymer is at least one selected from the group consisting of polyglycolic acid (PGA), polylactic acid (PLA), poly(lactic-co-glycolic acid) (PLGA), poly-ε-caprolactone (PCL), polyanhydride, polyorthoesters, polyvinylalcohol, polyethylene glycol, polyurethane, polyacrylic acid, poly-N-isopropyl acrylamide, poly(ethylene oxide)-poly(propylene oxide)-poly(ethylene oxide) copolymers, derivatives thereof, and copolymers thereof.

7. The composite scaffold of claim 5, wherein the human mesenchymal stem cells are derived from bone marrow.

8. A method for manufacturing the composite scaffold of claim 5, comprising:
1) electrospinning a 8˜15% solution of a biodegradable polymer in an organic solvent at a flow rate of 0.01˜5 mL/h to give an electrospun biodegradable polymer fibrous scaffold;
2) sterilizing the electrospun biodegradable polymer fibrous scaffold by immersing a disc of the electrospun biodegradable polymer fibrous scaffold of 1) in 50˜99% ethanol in a cell culture plate for 30˜100 min, followed by removing the organic solvent in a vacuum chamber for 2˜5 days;
3) immersing the sterilized electrospun biodegradable polymer fibrous scaffold in a complete growth medium supplemented with 15% FBS over the period of 48 hrs, followed by pipetting human mesenchymal stem cells, or chondrocytes or osteocytes that are differentiated from human mesenchymal stem cells, onto the electrospun biodegradable polymer fibrous scaffold, and by culturing the cells in a complete growth medium over the period of 24 hrs and then in a chondrogenic differentiation medium;
4) preparing a multiwalled carbon nanotube-phosphate buffered saline mixture by primarily ultrasonicating a mixture of multiwall carbon nanotubes, sulfuric acid, and nitric acid for 30˜100 min at 30˜70° C., neutralizing the mixture, centrifuging the mixture to collect the multiwalled carbon nanotubes, removing the solvents used, washing the multiwalled carbon nanotubes, secondarily ultrasonicating, recovering the multiwalled carbon nanotubes through centrifugation, and resuspending and dispersing the multiwalled nanotubes in phosphate buffered saline;
5) mixing 70% of collagen type II from articular cartilage, 6.5% of 10× HESS, 3.5% of 0.4 N NaOH, 1% of 0.4 N acetic acid, and 19% of sterile water to give a collagen hydrogel;
6) combining the multiwalled carbon nanotube-phosphate buffered saline mixture of 4) with the collagen hydrogel of 5), followed by adjusting the combination to pH of 7˜8 to give a multiwalled carbon nanotube-incorporated 3-D collagen type II-based hydrogel;
7) seeding and culturing either human mesenchymal stem cells or chondrocytes or osteocytes differentiated from human mesenchymal stem cells in the multiwalled carbon nanotube-incorporated 3-D collagen type II-based hydrogel of 6); and
8) pouring the cell-seeded, multiwalled carbon nanotube-incorporated 3-D collagen type II-based hydrogel of 7) to form a flat layer onto the cell-seeded electrospun, biodegradable polymer fibrous scaffold of 3), followed by allowing the hydrogel to completely set by incubation at 35˜40° C. for 30˜60 min.

9. The method of claim 8, wherein the biodegradable polymer is at least one selected from the group consisting of polyglycolic acid (PGA), polylactic acid (PLA), poly(lactic-co-glycolic acid) (PLGA), poly-ε-caprolactone (PCL), polyanhydride, polyorthoesters, polyvinylalcohol, polyethylene glycol, polyurethane, polyacrylic acid, poly-N-isopropyl acrylamide, poly(ethylene oxide)-poly(propylene oxide)-poly(ethylene oxide)copolymers, derivatives thereof, and copolymers thereof.

10. The method of claim 8, wherein the human mesenchymal stem cells are derived from bone marrow.

11. The method of claim 8, wherein the organic solvent is selected from the group consisting of methylene chloride, dimethyl formamide, hexane, chloroform, acetone, dioxane, tetrahydrofuran, hexafluoroisopropane, and a combination thereof.
